(19) United States
(12) Patent Application Publication
(10) Pub. No.: US 2004/0028675 A1
(43) Pub. Date: Feb. 12, 2004

(54) Compositions for the treatment of lupus

(75) Inventor: Randy H. Ziegler, Newport Coast, CA (US)

Correspondence Address: REED SMITH CROSBY HEAFEY LLP, 1901 AVENUE OF THE STARS, SUITE 700, LOS ANGELES, CA, 90067, US

(73) Assignee: Zarlink Semiconductor AB

(21) Appl. No.: 10/426,460

(22) Filed: Apr. 30, 2003

Related U.S. Application Data
(63) Continuation-in-part of application No. PCT/US02/39297, filed on Dec. 9, 2002.
(60) Provisional application No. 60/339,199, filed on Dec. 7, 2001.

(30) Foreign Application Priority Data
Apr. 30, 2002 (GB) 0209815.0

(51) Int. Cl. A61K 38/46; A61K 31/7048; A61K 31/375; A61K 31/353

(52) U.S. Cl. 424/94.65; 514/27; 514/456; 514/474

(57) ABSTRACT

A composition and a method for the amelioration of Lupus, related rheumatic and autoimmune diseases such as fibromyalgia and inflammatory joint diseases. The composition consists of a bioflavonoid combined with Bromelain. Vitamin C may be added to the composition to improve its efficacy. The preferred flavonoid is Luteolin or Quercetin. Myricetin may also be used as may be a glycoside such as Rutin that contains either Luteolin, Quercetin or Myricetin as an aglycone. Other effective flavonoids can be selected by their ability to interact with the Kv1.3 channel of lymphocytes. In a preferred method of treatment a mixture of flavonoid, Bromelain and Vitamin C is administered at least daily by an oral route. A mixture of 500 mg Quercetin, 500 mg Bromelain and 500 mg Vitamin C administered three times daily is effective.

The present application is a continuation in part of, and claims priority from, PCT/US02/39297 designating the United States which was based on and claimed priority from U.S. Provisional Patent Application No. 60/339,199 filed on Dec. 7, 2001.

### BACKGROUND OF THE INVENTION

1. Area of the Art

The present invention concerns a treatment for autoimmune rheumatic diseases and for joint diseases.

2. Description of the Prior Art

Lupus Erythematosus (“Lupus”) is a chronic inflammatory disease that can affect the skin, joints, blood, and kidneys as well as other parts of the body. Lupus is an “autoimmune” disease in which the immune system makes antibodies directed against parts of the body. Normally antibodies react only with bacteria, viruses and other foreign substances. When “self” antibodies are made, damage can occur either through direct antibody mediated attack on body tissues or indirectly from immune complexes. Immune complexes are the reaction products between portions of the body's tissues and the antibodies. These complexes build up in the skin or in joints or in kidneys and cause many of the symptoms of Lupus. Although many cases of Lupus are mild, the disease may cause serious life-threatening symptoms.

Lupus is one of a number of “rheumatic” diseases that generally also include an autoimmune component. Usually much of the damage of Lupus and other rheumatic diseases is mediated by inflammatory processes. Inflammation is generally a process wherein circulation to a region of tissue is increase and blood vessel walls become leaky so that white blood cells can infiltrate the region and attack foreign bodies. If autoantibodies are involved the bodies own tissues are attacked and damaged or destroyed. If the inflammation occurs in the joints, the cushioning and lubricating cartilage of the joints can be damaged. This causes frictional damage to the bones of the joint, which can result in more inflammation and more damage. This is why anti-inflammatory drugs are often used to treat Lupus and other autoimmune inflammatory and inflammatory joint diseases. It is generally believed that some joint diseases such as osteoarthritis are more of a “wear and tear” disease wherein the cartilage is naturally eroded. However, the end product is still inflammation (which causes further damage) so that anti-inflammatory drugs are also effective. Further there are a number of chronic autoimmune diseases such as fibromyalgia that, while generally not life threatening, result in a great deal of pain and discomfort.

The most commonly used anti-inflammatory drugs are non-steroidal anti-inflammatory drugs such as ibuprofen or ketoprofen which act on the inflammatory processes downstream of the white blood cells. Steroids directly affect the white blood cells by slowing their replication or b speeding their destruction through apoptosis. Other effective drugs are cytotoxic drugs such as Imuran which suppress the immune system (and hence inflammation) by damaging rapidly dividing cells such as immune cells.

As will be appreciated, all of these drug treatments can have serous side effects. Non-steroidal anti-inflammatory drugs can cause serious damage to the digestive system and often prolong bleeding times. Steroids can be adductive and have a wide range of serious side effects including diabetes. Cytotoxic drugs can result in oversuppression of the immune system with resulting infections and even cancer. Further, many inflammatory diseases—especially Lupus—may not respond adequately to any of the treatments. Consequently, there remains an extreme need for safe and effective treatments for Lupus and other autoimmune inflammatory diseases such as arthritis and fibromyalgia.

### SUMMARY OF THE INVENTION

The inventor has found that bioflavonoids, particularly flavanols such as Quercetin, supplemented with Bromelain and Vitamin C, (500 mg of each, in doses three times a day) completely reverse the symptoms of Lupus and other autoimmune and inflammatory joint diseases. Luteolin, Myricetin and Rutin are also effective bioflavonoids in the present invention. Experimental data indicate that Luteolin is somewhat more effective than Quercetin. Luteolin, however, is currently difficult to obtain economically in quantity.

While not wishing to be bound to a single explanation for the working of the present invention, the inventor believes that a significant part of the efficacy is due to the effects of specific bioflavonoids on certain cellular ion channels. In the past it has been shown that abnormal potassium channel expression in certain autoimmune diseases is related to the abnormal immune response. (See “Abnormal K⁺ channel expression in AutoImmune Diseases . . . ” by Chandy and Cahalan published in 1990 by the University of California at Irvine and “Autoimmune diseases linked to abnormal K⁺ channel expression in double-negative CD4⁻CD8⁻T cells,” Eur J. Immunol, Cahalan M D, Chandy G K, Grissmer S., 1990, 20: 747-751). It was shown rats with experimental SLE (Systemic Lupus Erythematosus), Diabetes (Type I) and EAE (Experimental Autoimmune Encephalitis) were tested for the Kv1.3 channel, and it was shown found that all three have the CD4 and CD8—T-Cells (double negative T-Cells) expression that inflames the various tissues associated with the respective diseases. All of these diseases show a control of lymphocyte proliferation by the Kv1.3 channel. It has independently been demonstrated that flavonoids, especially Quercetin, are very effective at modulating the conduction by this potassium channel. This suggested to the inventor that flavonoids could be effective in controlling human Lupus.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an effective treatment for Lupus and other autoimmune diseases and inflammatory joint diseases

Flavonoids are ubiquitous secondary products found in most land plants. Flavonoids are oxygen based rather than nitrogen based like traditional pharmaceuticals plant compounds such as alkaloids. As such they are end terminus electron acceptors rather than electron donors. Flavonoids inhibit 5-lipoxygenase in the cytokine release pathways because hydroxyl groups at 4′, 3, and 7 positions of the flavonoid molecules accept electrons. (See, Effects of FLAVONOIDS on Arachidonic Acid Metabolism, A. F. Welton, L. D. Tobias, The Biochemistry of Cell Activation Related to the Putative Action of FLAVONOIDS, S. G. Laychock, Plant Flavonoids in Biology and Medicine: Biochemical, Pharmacological, and Structure-Activity Relationships, pg 231-242, 1986 Alan R Liss, Inc.). Flavonoids modulate the immune response through sequestration of free radicals, which prevents formation of epoxide diols and subsequent attack on the DNA. Further, catalytic ion and signal transducers are sequestered by Flavonoids (See, Structural dependence of flavonoid interactions with Cu⁺⁺ ions: implications for their antioxidant properties, Brown J E, Khodr H, Hider R C, Rice-Evans C A, Biochem. J. Mar. 15, 1998;330 (pt 3): 1173-8).

A number of biological effects of flavonoids have been elucidated. For example, flavonoids inhibit Deiodinase, which is the enzyme that promulgates thyroid functions. Iodothyronine deiodinase is oxygen bound and directly impacts basal oxygenation. Flavonoids have the steric binding mimicry of ligands that bind the oxygen transport molecule Iodothreonine Deiodinase. (“Role of FLAVONOIDS in the Oxygen-free Radical Modulation of Immune Responses”, B. Pignol, et. al., Role of Flavonoids in the Oxygen-Free Radical Modulation of the Immune Response, Plant Flavonoids in Biology and Medicine II: Biochemical, Cellular, and Medicinal Properties, pg. 173-182, 1988 Alan R. Liss, Inc.; “Structure Activity Relationships of FLAVONOIDS Deiodinase Inhibitors and Enzyme Active Site Model”, S. V. Cody, Plant Flavonoids in Biology and Medicine: Biochemical, Pharmacological, and Structure-Activity Relationships, 1986 Alan R. Liss, Inc., pg. 373-382). Flavonoids thus “turn down the thermostat” of homeostasis. (“Iodothyronine Deiodinase is Inhibited by Plant FLAVONOIDS”, J. Koehrle pg. 324, Structure Activity Relationships of FLAVONOIDS Deiodinase Inhibitors and Enzyme Active Site Model, S. V. Cody, Plant Flavonoids in Biology and Medicine: Biochemical, Pharmacological, and Structure-Activity Relationships, 1986 Alan R. Liss, Inc., pg. 373-382).

It is believed that the effect of flavonoids in the present invention is mediated through the above-discussed effect on ion channels. However, the effect on the thyroid indicates that the flavonoids may be acting on more than one biological system in the present invention. The inventor included Bromelain in the present invention because there is some indication that this enzyme can aid in the regeneration of cartilage in a damaged joint. Generally, Bromelain alone is not effective in ameliorating the joint pain in either osteoarthritis or Lupus caused joint inflammation. It is logical that the compound is not effective in Lupus because continued Lupus-induced inflammation would mask any joint repairing effects of Bromelain. It also seems apparent that osteoarthritis, at least in some cases, is mediated by an inflammatory process. This may explain why Bromelain treatment of that disease has not been very effective.

However, the flavonoid component of the present invention controls inflammation through its effects on ion channels and possibly through other biological effects not yet elucidated. White blood cells are deactivated by the changes in ion channels so that inflammatory attacks on the joints are decreased. Down regulation of the immune system (as mediated by white blood cells) results in lowered production of autoantibodies. Under these conditions Bromelain appears to be able to help in joint repair. It does seem likely that Bromelain is in some way synergistic with the flavonoids because treatment with flavonoids but without Bromelain is clearly less effective than the combination. Many natural foods contain flavonoids; however, few, if any, contain Bromelain combined with effective flavonoids. Also, it is not known whether the combination of flavonoids in natural products might decrease the overall effectiveness of the “correct” flavonoids. For whatever reasons mere dietary treatments for Lupus and related diseases has not been successful. While the present inventor believes that the addition of vitamin C to the flavonoid/Bromelain mixture enhances the effect of the mixture, it is apparent that the mixture without vitamin C is also effective. However, because vitamin C is relatively innocuous, it has been used in the vast majority of tests.

Screening tests by experts in ion channel function have indicated that a number of flavonoids, especially certain flavanols have the correct charge structure to regulate the key ion channels. Quercetin, which has been used in the majority of tests of the present invention, is very effective in tests of ion channel function. Luteolin is even more effective than Quercetin in such tests. Limited tests have shown that replacing Quercetin with Luteolin in the inventive formula is somewhat more effective. However, at this time the inventor lacks a ready source of pharmaceutical grade Luteolin to demonstrate whether luteolin is significantly more effective overall. Quercetin and Luteolin differ by a single hydroxyl group (which Luteolin lacks as compared to Quercetin). A limited number of tests have shown that Myricetin is somewhat effective in the present; this compound varies from Quercetin in the addition of one hydroxyl. In addition, rutin is also moderately effective in the present invention. Rutin is a glycoside (rutinoside) of Quercetin so that metabolism of Rutin is likely to make Quercetin available. It is likely that other flavonoids with structural similarities to quercetin or luteolin, or glycosides of these flavonoids, will also function in the present invention.

The results of treating actual patients with the inventive composition have been extremely dramatic. Besides the cases described herein literally dozens of patients have shown similar results when treated with the Quercetin/Bromelain/Vitamin C mixture.

One of the first tested subjects was, “A”, who had been bed ridden for nine months and could neither sit up or turn over. She showed the typical Lupus butterfly rash and was diagnosed by a blood test showing a positive ANA (anti-nucleic acid antibody) titer—criteria indicative of Lupus. The subject's mother had previously tested positive for Lupus and has been treated with several medications, including Plaquinel and Cyclosporin. A second subject, “B”, had also tested positive for Lupus and had been being treated with Plaquinel and Cyclosporin.

Both of these test subjects experienced rapid relief from symptoms of fatigue and depression and pain, elimination of the associated rash and a return to normal life style within one to two months of beginning a regime of one pill (500 mg Quercetin/500 mg Bromelain/500 mg Vitamin C) three times per day at the above dosage. Kidney flares ceased and pain abrogated. Both subjects stated that they felt completely fine with no side effects and maintained a perfect state of health while on the inventive composition.

Both A and B stopped taking the medication after two months, since they reported that they were “completely recovered.” As might be expected, symptoms begin to reappear within two weeks. Upon reestablishment of treatment all symptoms again disappeared. This indicates that the inventive product is solely responsible for the amelioration of SLE (Systemic Lupus Erythematosus).

Additional patients have also shown similar positive results. “C” was a 65-year-old woman with seropositive rheumatoid arthritis. She developed side effects from methotrexate treatment at a dose of 22.5 mg weekly. Her methotrexate was discontinued and she was started on the inventive composition. Within one month she showed a dramatic decrease in synovitis.

“D” was a 37-year-old woman with an inflammatory polyarthritis and positive ANA (antinuclear antibody). She had Sjogrens syndrome with a positive SSA (anti-Ro) antibody. After three months of treatment with the inventive composition, she noticed decreased hair loss, and a lessening of joint pain and swellings. Her serologies also improved. Her initial ANA titer was 170 and SSA titer was 584, and occasionally much, much higher. Her most recent serologies show an ANA titer of 67 and SSA of 429.

“E” was a 57 year old female with SLE/Sjogrens. Her disease manifestations were pulmonary infiltrates and pulmonary effusions. Despite high doses of prednisone, plaquenil, and Imuran she had continued pulmonary symptoms, fevers, joint swellings, and elevated anti-DNA antibody. After one-month treatment with the inventive composition, her Anti-DNA level decreased from 454 to 186 (normal less than 30).

“F” was a 46 year old female with SLE. Her main manifestations have been joint inflammation and recurrent serositis. She has had recurrent chest pain and palpitations. Her echocardiogram in 1999 showed valvular changes with trace mitral regurgitation and mild to moderate tricuspid regurgitation. She also had pulmonary hypertension with a RV systolic pressure of 45. “F” started the inventive composition and after four months was tapered off prednisone of 20-mg qd and Plaquinel. Her recent echocardiogram shows improvement—there was no longer any evidence of pulmonary hypertension.

“G” was a 35-year-old woman who had suffered from had SLE and fibromyalgia for the past several years. Her current medications are Levoxyl, Vioxx 25 mg/d and the inventive composition. G had been unable to tolerate any traditional medications because of side effects. However, with the inventive composition she reported significant decreases in fatigue and pain. Objectively she showed decreased joint tenderness and fibromyalgia tender points—improvements not attained without the inventive composition.

“H” was a 38 year old woman with SLE and Sjogrens syndrome. She started the inventive composition and within nine months she had significantly decreased joint pains and swellings. Joint symptoms are now nearly resolved. Objectively she has no joint tenderness or inflammation, her erythrocyte sedimentation rate (ESR) decreased from 44 to 26 over an eight month period.

Patient “I” was a 65 year old woman who had suffered from rheumatoid arthritis for several years. Prior to use of the inventive composition she was being treated with methotrexate at 12.5 mg/week, Mobic at 15 mg/day. The inventive composition was added and I exhibited decreased joint pains and swellings and a decreased number of inflamed joints. Her ESR levels decreased from 79 to 28 over a four and one half month treatment period.

Patient “J” was a 64 year old woman who had SLE for several years. Current medications were prednisone at 7 mg/d, and plaquinel at 400 mg/d, The inventive composition was started and within six months the patient showed decreased joint pain and was able to lower her prednisone dose. She stopped taking the inventive composition and developed an arthritis flare. She started taking the inventive composition again after three months, and her Anti-ds-DNA antibody level decreased.

Patient “K” was a 50 year old woman with a several year history of SLE and fibromyalgia. Her current medications are the inventive composition, plaquenil 400 mg/d, lortab prn, levoxyl, and duragesic pain patch. Within three months of adding the inventive composition to her medications the patient experience a significant decrease in both pain and fatigue.

At this time more than 50 patients, most with some form of Lupus, but others with various autoimmune diseases, such as fibromyalgia, and inflammatory joint diseases, have been treated with the inventive composition. Virtually all patients have demonstrated a measurable amelioration of their disease state.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

I claim:

1. A composition for the treatment of Lupus, autoimmune diseases and inflammatory joint diseases comprising flavonoid and Bromelain.

2. The composition according to claim 1 further comprising Vitamin C.

3. The composition according to claim 1, wherein the flavonoid is selected from the group consisting of Luteolin, Quercetin, Myricetin and Rutin.

4. The composition according to claim 1, wherein the flavonoid comprises a flavonol.

5. The composition according to claim 1, wherein the flavonoid comprises a glycoside.

6. The composition according to claim 5, wherein the glycoside has an aglycone that is a flavonol.

7. The composition according to claim 6, wherein the flavonol is selected from the group consisting of Quercetin, Luteolin and Myricetin.

8. The composition according to claim 1, wherein the flavonoid is selected to interact with lymphocyte Kv1.3 channels.

9. The composition according to claim 6, wherein the flavonol interacts with lymphocyte Kv1.3 channels.

10. The composition according to claim 1, wherein the flavonoid comprises Quercetin.

11. The composition according to claim 10 further comprising Vitamin C.

12. A method for the treatment of Lupus, autoimmune diseases and inflammatory joint diseases in a patient comprising the step of repeatedly administering flavonoid and Bromelain.

13. The method according to claim 12, wherein the step of administering further comprises administering Vitamin C.

14. The method according to claim 12, wherein the step of administering is oral administration.

15. The method according to claim 14, wherein the step of administering is repeated at least daily.

16. The method according to claim 15, wherein the step of administering is repeated at least twice daily.

17. The method according to claim 12, wherein the flavonoid is selected from the group consisting of Luteolin, Quercetin, Myricetin and Rutin.

18. The method according to claim 12, wherein the flavonoid comprises a flavonol.

19. The method according to claim 12, wherein the flavonoid comprises a glycoside.

20. The method according to claim 19, wherein the glycoside has an aglycone that is a flavonol.

21. The method according to claim 20, wherein the flavonol is selected from the group consisting of Quercetin, Luteolin and Myricetin.

22. The method according to claim 12, wherein the flavonoid is selected to interact with lymphocyte Kv1.3 channels.

23. The method according to claim 20, wherein the flavonol interacts with lymphocyte Kv1.3 channels.

24. The method according to claim 12, wherein the flavonoid comprises Quercetin.

25. The method according to claim 24 further comprising Vitamin C.

26. A method for the treatment of Lupus, autoimmune diseases and inflammatory joint diseases in a patient comprising the step of repeatedly administering a mixture of Quercetin, Bromelain and Vitamin C.

27. A composition for the treatment of Lupus, autoimmune diseases and inflammatory joint diseases comprising a mixture of Quercetin, Vitamin C and Bromelain.
